(19) United States
(12) Patent Application Publication
(10) Pub. No.: US 2015/0359923 A1
(43) Pub. Date: Dec. 17, 2015

(54) Method Of Manufacturing Biodegradable Materials For Filling Nasal Cavity

(71) Applicants: Keng-Liang Ou, Taipei City (TW); CHIH-WEN WANG, New Taipei City (TW)

(72) Inventors: Keng-Liang Ou, Taipei City (TW); CHIH-WEN WANG, New Taipei City (TW)

(73) Assignee: MONITEX INDUSTRIAL CO., LTD., New Taipei City (TW)

(21) Appl. No.: 14/304,968

(22) Filed: Jun. 15, 2014

(51) Int. Cl. A61L 15/22; A61L 15/44; A61L 15/64

(52) U.S. Cl. CPC A61L 15/225; A61L 15/64; A61L 15/44; A61L 2300/252; A61L 2300/236; A61L 2300/406; A61L 2300/412

(57) ABSTRACT

A method of manufacturing biodegradable materials for filling nasal cavity of the present invention comprises the following steps: a. within a predetermined temperature range, evenly mixing chitosan and the substrate (starch or cellulose) to make a first solution; b. adding crosslinking agent in the first solution for crosslinking reaction; c. pouring the first solution into the mold; d. removing water through freeze-drying process, thereby the biodegradable materials for filling nasal cavity are obtained. The biodegradable materials are filled in patient's nasal cavity to stop bleeding and avoid a second time injury by removing, because of biodegradable absorption.

### FIELD OF THE INVENTION

The present invention relates to a method of manufacturing biodegradable materials for filling nasal cavity; particularly to a method of manufacturing biodegradable materials which are used to fill nasal cavity to stop bleeding and avoid a second time injury by removing, and which at the same time accelerate wound healing.

### BACKGROUND OF THE INVENTION

Respiratory diseases are common modern illnesses, inclusive of respiratory allergies, allergic rhinitis and sinusitis, if these diseases get worse or cause extreme discomfort in patients, it is necessary to use drug therapy or rhinological surgery.

After rhinological surgery, the traditional approach is to use vaseline gauze and Iodoform to stop bleeding, but such kind of filling materials are difficult to remove. By removing filled materials it is often painful for patients, and makes easily second time injury caused by nasal injury.

In view of these disadvantages the inventor tried the continuous testing and improvement and developed the present invention.

### SUMMARY OF THE INVENTION

The main object of the present invention is to provide a method of manufacturing biodegradable materials for filling nasal cavity to avoid a second time injury by removing.

For achieving above object, the method of manufacturing biodegradable materials for filling nasal cavity of the present invention comprises the following steps: a. Within a predetermined temperature range, evenly mixing chitosan and the substrate (starch or cellulose) to make a first solution; b. Adding crosslinking agent to the first solution for crosslinking reaction; c. Pouring the first solution into the mold; d. Removing water through freeze-drying process, thereby the biodegradable materials for filling nasal cavity are obtained. The biodegradable materials are filled in patient's nasal cavity to stop bleeding and avoid a second time injury by removing, because of biodegradable absorption.

Another object of the present invention is to provide a method of manufacturing biodegradable materials for filling nasal cavity to accelerate wound healing.

For achieving above object, the present invention is characterized thereby: adding a second solution (Collagen and/or an immunomodulatory protein from Ganoderma Lucidum (Ling Zhi-8 or abbreviated as LZ-8)) in the first solution, so that wound healing is accelerated.

Other aspects and advantages of the present invention will become apparent from the following detailed description, taken in conjunction with the accompanying drawing, illustrating by way of example the principles of the present invention.

### BRIEF DESCRIPTION OF THE DRAWINGS

FIG. 1 is a flow chart of manufacturing of the present invention.

### DESCRIPTION OF THE PREFERRED EMBODIMENTS

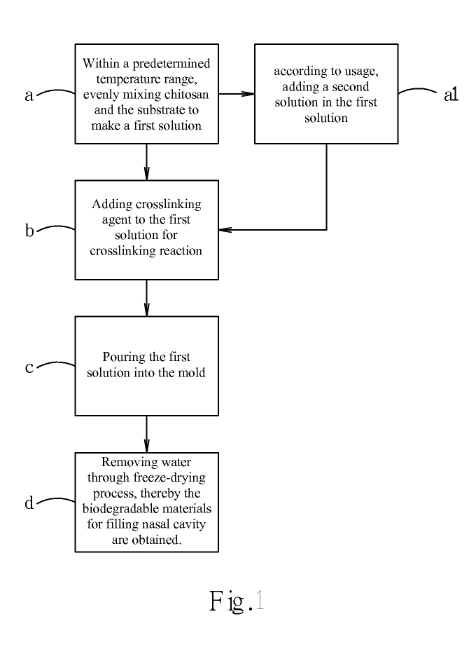

As shown in FIG. 1, a method of manufacturing biodegradable materials for filling nasal cavity of the present invention comprises the following steps:

a. Within a predetermined temperature range, evenly mixing chitosan and the substrate to make a first solution; b. Adding crosslinking agent in the first solution for crosslinking reaction; c. Pouring the first solution into the mold; d. Removing water through a freeze-drying process, thereby the biodegradable materials for filling nasal cavity are obtained.

In the step a, within a predetermined temperature range, evenly mixing chitosan and the substrate to make a first solution, one can choose biodegradable materials like Starch or Methyl cellulose to use as the substrate, to avoid a second time injury by removing.

Adding Chitosan gives antibacterial activity, wherein the predetermined temperature range is 80 to 100° C. In this temperature range it helps evenly mixing Chitosan and substrate. Besides, the Chitosan comprises 30-50 total weight percent, the substrate 30-50 total weight percent and the crosslinking agent 10-30 total weight percent, furthermore, a preferred ratio of the chitosan and the substrate is 1:1.

According to usage, one can firstly process al after the step a has been done, adding a second solution in the first solution, thereafter process the step b. One can choose the materials to accelerate wound healing, like Collagen and/or LZ-8, to use as the said second solution. Adding collagen is effective for enhancing tissue binding and LZ-8 for increasing the resistance and easing allergies. Besides, the Chitosan comprises 20˜40 total weight percent, the substrate 20˜40 total weight percent, the Collagen 10˜20 total weight percent, LZ-8 10˜20 total weight percent and the crosslinking agent 5˜15 total weight percent. Furthermore a preferred ratio of the chitosan, the substrate, the Collagen and LZ-8 is 2:2:1:1. By adding Collagen (or LZ-8) alone, the Chitosan comprises 20˜40 total weight percent, the substrate 20˜40 total weight percent, the Collagen 10˜20 (or LZ-8) 20˜40 total weight percent and the crosslinking agent 5˜15 total weight percent. Furthermore a preferred ratio of the chitosan, the substrate and the Collagen (or LZ-8) is 1:1:1.

Step b is adding crosslinking agent in the first solution for crosslinking reaction, which builds a porous structure. Simulated experiments of the present invention were made with samples 1,2, the ratio of the components is shown in annex 1. As shown in the annex 2, as the samples 1 and 2 are filled in patient's wound as a support to avoid tissue collapse, a porous structure is conducive to the growth of cells clinging. As shown in annexes 3˜4, the porosity of the biodegradable materials for filling nasal cavity of the present invention reaches 98˜99%. As crosslinking agent one can choose materials like Cidex for building a porous structure of Chitosan and substrate, wherein a preferred ratio of the Chitosan, substrate, Collagen, LZ-8 and Cidex is 6:6:3:3:2.

Step c is pouring the first solution into a mold. Since the conditions of wound are different, the biodegradable materials for filling nasal cavity of the present invention can be formed spherical or other shapes.

Step d is removing water through freeze-drying process. The biodegradable materials for filling nasal cavity having a porous structure of a sponge-like feature formed by cross-linking reaction, can be filled in patient's wound to stop bleeding and prevent collapse of surrounding tissue. Since its composition is manufactured by biodegradable materials, thus after filling it dose not need to be removed, thereby a second time injury is avoided. Furthermore, according to usage, one can add Collagen and/or LZ-8 to accelerate healing wound.

Besides, the hydrophility of the filling materials affects the efficiency of blood absorption. When the filling materials are filled in patient's wound, blood absorption stops bleeding. Therefore the filling materials are preferred to be hydrophilic. In the test of contact angles, if the filling materials have a contact angle under 90 grade, the hydrophility is well. As shown in annexes 5˜6, using water, physiological saline and blood for contact angles test of the present invention, it results that, the biodegradable materials for filling nasal cavity of the present invention are hydrophilic and efficient for blood absorption. When the biodegradable materials are filled in patient's wound, will be quickly inflated through absorption and thereby bleeding is stopped.

Moreover, it is very important, if the filling materials generate Cytotoxin. As shown in the annexes 7˜8, in the MTT assay, MG63 and NIH3T3 are used for cytotoxicity test of the present invention, wherein the negative control group is HDPE extract generating no Cytotoxin; the positive control group is phenol diluent generating Cytotoxin. It results, that the biodegradable materials for filling nasal cavity of the present invention can not generate Cytotoxin.

For describing the biodegradation process of the biodegradable materials for filling nasal cavity of the present invention, the rabbit sinus implantation experiment, as shown in annexes 9˜10, is used as an example.

Annexes 9˜10 are SEM scan pictures from the rabbit sinus implantation experiments with sample 2. As shown in annex 9, one week after surgery the sample 2 is still found. In the picture around the dotted line it is observed that tissue is darker and inflamed.

As shown in the annex 10, four weeks after surgery it is observed that the sample 2 causes angiogenesis within the damaged tissue and is clearly thickening (arrows of the dotted lines). Additionally, around the damaged bone tissue there are osteoblasts which helps repair.

1. A method of manufacturing biodegradable materials for filling nasal cavity, comprises the following steps:
a. within a predetermined temperature range, evenly mixing chitosan and the substrate to make a first solution;
b. adding crosslinking agent to the first solution for crosslinking reaction;
c. pouring the first solution into the mold; and
d. removing water through freeze-drying process, thereby the biodegradable materials for filling nasal cavity are obtained.

2. The method of manufacturing biodegradable materials for filling nasal cavity of claim 1, wherein the said predetermined temperature range in step a is 80 to 100° C.

3. The method of manufacturing biodegradable materials for filling nasal cavity of claim 1, wherein the said substrate in step a is starch.

4. The method of manufacturing biodegradable materials for filling nasal cavity of claim 1, wherein the said substrate in step a is Methyl cellulose.

5. The method of manufacturing biodegradable materials for filling nasal cavity of claim 1, wherein the said Chitosan in step a comprises 30˜50 total weight percent, the said substrate 30˜50 total weight percent and the said crosslinking agent 10˜30 total weight percent; the ratio of the chitosan and the substrate is 1:1.

6. The method of manufacturing biodegradable materials for filling nasal cavity of claim 1, wherein after processing step a processes a step al: adding a second solution in the first solution, thereafter processes the step b.

7. The method of manufacturing biodegradable materials for filling nasal cavity of claim 6, wherein the second solution is Collagen.

8. The method of manufacturing biodegradable materials for filling nasal cavity of claim 7, wherein the Chitosan comprises 20˜40 total weight percent, the substrate 20˜40 total weight percent, the Collagen 20˜40 total weight percent, and the crosslinking agent 5˜15 total weight percent.

9. The method of manufacturing biodegradable materials for filling nasal cavity of claim 6, wherein the second solution is Ling Zhi-8.

10. The method of manufacturing biodegradable materials for filling nasal cavity of claim 9, wherein the Chitosan comprises 20˜40 total weight percent, the substrate 20˜40 total weight percent, the Ling Zhi-8 20˜40 total weight percent, and the crosslinking agent 5˜15 total weight percent.

11. The method of manufacturing biodegradable materials for filling nasal cavity of claim 6, wherein the second solution is a mixed solution of collagen and Ling Zhi-8.

12. The method of manufacturing biodegradable materials for filling nasal cavity of claim 11, wherein the Chitosan comprises 20˜40 total weight percent, the substrate 20˜40 total weight percent, the collage 10˜20 total weight percent, the Ling Zhi-8 10˜20 total weight percent, and the crosslinking agent 5˜15 total weight percent.

13. The method of manufacturing biodegradable materials for filling nasal cavity of claim 12, wherein the said crosslinking agent in step b is Cidex.
